(19) United States
(12) Patent Application Publication
(10) Pub. No.: US 2004/0229251 A1
(43) Pub. Date: Nov. 18, 2004

(54) Detection of guanylyl and adenylyl cyclase activity

(75) Inventors: Roger K. Sunahara, Ann Arbor, MI (US); Richard R. Neubig, Ann Arbor, MI (US)

Correspondence Address: Tanya A. Arenson, MEDLEN & CARROLL, LLP, Suite 350, 101 Howard Street, San Francisco, CA, 94105, US

(73) Assignee: The Regents of the University of Michigna, Ann Arbor, MI (US)

(21) Appl. No.: 10/779,404

(22) Filed: Feb. 13, 2004

Related U.S. Application Data
(60) Provisional application No. 60/447,074, filed on Feb. 13, 2003.

(51) Int. Cl. C12Q 1/68; C12N 9/22

(52) U.S. Cl. 435/6; 435/199

(57) ABSTRACT

The present invention relates to methods of assaying nucleotide cyclase activity. In particular, the present invention relates to fluorescence-based methods of assaying guanylyl and adenylyl cyclase activity.

This application claims priority to provisional patent application serial No. 60/447,074, filed Feb. 13, 2003, which is herein incorporated by reference in its entirety.

This invention was made with government support under Grant No. GM39565 awarded by the National Institutes of Health. The Government has certain rights in the invention.

### FIELD OF THE INVENTION

The present invention relates to methods of assaying nucleotide cyclase activity. In particular, the present invention relates to fluorescence-based methods of assaying guanylyl and adenylyl cyclase activity.

### BACKGROUND OF THE INVENTION

Nucleotide cyclases such as adenylyl cyclases (AC) and guanylyl cyclases (GC) produce cyclic nucleotides. GC activity has been directly implicated in the vasodilatory effect of nitric oxide, as well as the diuretic effect of Atrial Natriuretic Factor. The enzyme contributes to production of cyclic nucleotides in retina and is intimately involved in the process of vision. The natural ligands for only four of eight known membrane-bound GC isoforms have been identified and are solely responsible for the above-mentioned activities. The search for novel synthetic ligands for the known isoforms and the natural ligands for the remaining four mammalian isoforms has been the focus of the pharmaceutical biotechnology industry. The search for ligands would greatly benefit from the development of a high throughput screening process.

ACs are involved in numerous signaling pathways ranging from regulation of glycogen metabolism to cell differentiation and to memory and long-term potentiation. Unlike GCs, which contain a receptor within their coding region, ACs are regulated by cell surface hormone receptors that couple to G proteins. There are nine membrane-bound isoforms that are the targets for regulation mainly by G proteins. While there are several modes of regulation of AC activity, all modes target the intracellular catalytic domains. Little is currently known about the transmembrane and ecto-domains. The potential of ACs as therapeutic targets has only recently been appreciated due to the human genome project as well as understanding of AC function. Reports of the development of isoform-specific inhibitors are only now beginning to surface, however the efficacies are far from being therapeutically usefulness. The discovery of more selective and more potent compounds would greatly benefit from implementation of a high throughput screening assay.

Currently two methods are commonly used to measure guanylyl and adenylyl cyclase activity. One method utilizes radioactive GTP (or ATP) and measures the accumulation of radioactive cGMP (or cAMP), while the other method uses a radioimmunoassay procedure (RIA) to detect cGMP (or cAMP). For example, one assay measures the conversion of radioactive GTP (or ATP) to radioactive cGMP (or cAMP). For intact cell studies the conversion of metabolically-labeled [³H]GTP or [³H]ATP, derived from whole cells assays incubated with [³H]guanine, or [3H]adenine) to [³H]cGMP (or [³H]cAMP) is measured. For broken cell assays or assays with purified GC protein, the conversion of [³²P]αGTP to [³²P]cGMP, or [³²P]αATP to [³²P]cAMP, is measured. Following activation of the enzyme, the reaction is terminated by acid treatment, SDS solublization or by heat inactivation. In either case radioactive cGMP is separated from other radioactive species, a lengthy process that involves multiple chromatography steps. Another method measures non-radioactive cGMP levels using antibodies against cGMP in a radioimmunoassay (RIA). This process requires lengthy incubations and still requires the use of radioactivity, usually [¹²⁵I]cGMP, as a tracer.

These methods utilize lengthy protocols all requiring the use of radioactive compounds. GC assays using either protocol require several hours to obtain results and data cannot be attained in a real-time format. Furthermore, multiple transfers during the isolation of cGMP and scintillation counting, Cerenkov detection, or gamma radiation detection are required. All of these processes result in significant accumulation of radioactive wastes. In order to screen potential AC and GC ligands and modulators, additional screening methods are needed. Preferred assays are those suitable for high throughput screening.

### SUMMARY OF THE INVENTION

The present invention relates to methods of assaying nucleotide cyclase activity. In particular, the present invention relates to fluorescence-based methods of assaying guanylyl and adenylyl cyclase activity.

For example, in some embodiments the present invention provides a method comprising providing a nucleotide cyclase enzyme, a fluorescently labeled substrate for the nucleotide cyclase enzyme, and one or more test compounds; and contacting the nucleotide cyclase enzyme with the fluorescently labeled substrate and the test compound; and determining the level of fluorescence of the fluoresently labeled substrate. In some embodiments, the method further comprises the step of comparing the level of fluorescence of the fluorescently labeled substrate in the presence of the test compound to the level of the fluorescence in the absense of the test compound. In some embodiments, the level of fluorescence is increased in the presence of the test compound relative to in the absense of the test compound. In some embodiments the nucleotide cyclase enzyme is a guanylyl cyclase. In other embodiments, the nucleotide cyclase enzyme is an adenylyl cyclase enzyme. In other embodiments, the cyclase is a soluble cyclase. In still further embodiments, the nucleotide cyclase enzyme is an orphan receptor. In yet other embodiments, the nucleotide cyclase enzyme is membrane bound.

In some embodiments, the fluorescently labeled substrate is fluorescently labeled GTPγS (e.g., BODIPY FL-GTPγS). In some embodiments, the method further comprises the step of providing an activator of the nucleotide cyclase. In some embodiments, the activator is forskolin. In some embodiments, the nucleotide cyclase in a VC1-ACGC mutant of type II adenylyl cyclase. In some embodiments, the method further comprises the step of providing Mn²⁺.

In some embodiments, the contacting and the determining are performed in a multi-well microtitre plate. In some preferred embodiments, the method is a high-throughput method (e.g., a method for performing at least 96, preferably at least 384, and even more preferably at least 1536 simultaneous reactions).

In some embodiments, the test compound is a suspected ligand of the nucleotide cyclase enzyme. In some embodiments, the test compound is a suspected activator of the nucleotide cyclase enzyme. In some embodiments, the test compound is a drug. In some embodiments, the test compound is a suspected inhibitor of the nucleotide cyclase enzyme.

The present invention further provides a kit, comprising a nucleotide cyclase enzyme; and a fluorescently labeled substrate for the nucleotide cyclase enzyme. In some embodiments, the kit further comprises instructions for using the kit for screening one or more test compounds. In some embodiments, the test compound is a drug. In some embodiments, the test compound is a suspected activator or inhibitor of the nucleotide cyclase enzyme. In some embodiments, the nucleotide cyclase enzyme is an orphan receptor. In some embodiments, the test compound is a suspected ligand of the nucleotide cyclase enzyme.

In some embodiments, the kit further comprises an activator of the nucleotide cyclase (e.g., forskolin). In some embodiments, the kit further comprises Mn²⁺. In some embodiments, the nucleotide cyclase is an adenylyl cyclase, while in other embodiments, the nucleotide cyclase is a guanylyl cyclase. In some embodiments, the nucleotide cyclase is a soluble nucleotide cyclase. In some embodiments, the nucleotide cyclase in a VC1-ACGC mutant of type II adenylyl cyclase.

In some embodiments, the fluorescently labeled substrate is fluorescently labeled GTPγS (e.g., BODIPY FL-GTPγS). In some embodiments, the kit further comprises control nucleotide cyclase enzymes.

### DESCRIPTION OF THE FIGURES

FIG. 1 shows the fluorescence-based cyclase assay of the present invention. FIG. 1A illustrates the time-dependent, linear increase in fluorescence following activation of the purified, mutant adenylyl cyclase (VC1-ACGC) with activator, forskolin (Fsk). The effect of the mutation alters substrate specificity such that the enzyme utilizes GTP. FIG. 1B shows a catalytically inactive mutant of VC1 containing point mutation D396A is also inactive using BODIPY FL-GTPγS as a substrate.

FIG. 2 illustrates the effect of Mn²⁺ on guanylyl cyclase activity using BODIPY FL-GTPγS as a substrate. FIG. 2A shows fluorescence from 1 μM concentrations of VC1 and ACGC domains incubated in the presence of 5 mM Mg²⁺ and 5 mM Mn²⁺ (squares) or 10 mM Mg²⁺ (circles) in the absence (closed symbols), or presence (open symbol) of 100 μM forskolin (Fsk). FIG. 2B shows fluorescence from increasing concentrations of VC1 (100 pM to 3 μM) incubated with 100 nM of ACGC in the presence of 100 μM forskolin and 5 mM Mg²⁺ and 5 mM Mn²⁺. FIG. 2C shows that the affinity of the cyclase domains is unaltered using BODIPY FL-GTPγS as a substrate.

FIG. 3 illustrates that wild type adenylyl cyclase can use BODIPY FL-GTPγS as a substrate.

FIG. 4 describes the application of the fluorescence cyclase assay of some embodiments of the present invention to high throughput screening.

### DEFINITIONS

To facilitate understanding of the invention, a number of terms are defined below.

As used herein, the term “nucleotide cyclase enzyme” refers to any enzyme that converts a nucleotide triphosphate into a cyclic nucleotide. Exemplary nucleotide cyclases include, but are not limited to mammalian adenylyl cyclases and guanylyl cyclases, as well as cyclases from Bacillus anthrasis, mycobacteria, Rhizobia, yeast, paramecium, trypanosoma, etc.).

As used herein, the term “a fluorescently labeled substrate for said nucleotide cyclase enzyme” refers to any substrate (e.g., a nucleotide triphosphate) that can be utilized by a nucleotide cyclase enzyme as a substrate for conversion into the cyclic nucleotide and that is labeled with a fluorescent label. Any fluorescent label that does not interfere with the activity of the nucleotide cyclase enzyme may be utilized. Preferred labels are those that exhibit an alteration in fluorescence intensity (e.g., a dequenching) upon cyclization of the substrate. Exemplary labels include, but are not limited to, BODIPY.

As used herein, the term “determining the level of fluorescence of said fluorescently labeled substrate” refers to the quantitation of fluorescence signal from a fluorescent label attached to a substrate for a nucleotide cyclase enzyme. The increase in fluorescence is due to release of label from substrate.

As used herein, the term “activator of said nucleotide cyclase” refers to any agent that increases the cyclase activity of a nucleotide cyclase enzyme. Exemplary activators include, but are not limited to, nitric acid or forskolin.

As used herein, the term “host cell” refers to any eukaryotic cell (e.g., mammalian cells, avian cells, amphibian cells, plant cells, fish cells, and insect cells), whether located in vitro or in vivo.

As used herein, the term “mimetic” refers to a small molecule compound that mimics the binding of a ligand to its target.

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro, including oocytes and embryos.

As used herein, the term “vector” refers to any genetic element, such as a plasmid, phage, transposon, cosmid, chromosome, virus, virion, etc., which is capable of replication when associated with the proper control elements and which can transfer gene sequences between cells. Thus, the term includes cloning and expression vehicles, as well as viral vectors.

As used herein, the term “genome” refers to the genetic material (e.g., chromosomes) of an organism.

The term “gene” refers to a nucleic acid (e.g., DNA or RNA) sequence that comprises coding sequences necessary for the production of a polypeptide or precursor (e.g., proinsulin). The polypeptide can be encoded by a full length coding sequence or by any portion of the coding sequence so long as the desired activity or functional properties (e.g., enzymatic activity, ligand binding, signal transduction, etc.) of the full-length protein or fragment are retained. The term also encompasses the coding region of a structural gene and includes sequences located adjacent to the coding region on both the 5′ and 3′ ends for a distance of about 1 kb or more on either end such that the gene corresponds to the length of the full-length mRNA. The sequences that are located 5′ of the coding region and which are present on the MRNA are referred to as 5′ untranslated sequences. The sequences that are located 3′ or downstream of the coding region and which are present on the MRNA are referred to as 3′ untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The MRNA functions during translation to specify the sequence or order of amino acids in a nascent polypeptide.

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of MRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

Where “amino acid sequence” is recited herein to refer to an amino acid sequence of a naturally occurring protein molecule, “amino acid sequence” and like terms, such as “polypeptide” or “protein” are not meant to limit the amino acid sequence to the complete, native amino acid sequence associated with the recited protein molecule.

As used herein, the term “selectable marker” refers to a gene that encodes an enzymatic activity that confers the ability to grow in medium lacking what would otherwise be an essential nutrient (e.g. the HIS3 gene in yeast cells); in addition, a selectable marker may confer resistance to an antibiotic or drug upon the cell in which the selectable marker is expressed. Selectable markers may be “dominant”; a dominant selectable marker encodes an enzymatic activity that can be detected in any eukaryotic cell line. Examples of dominant selectable markers include the bacterial aminoglycoside 3′ phosphotransferase gene (also referred to as the neo gene) that confers resistance to the drug G418 in mammalian cells, the bacterial hygromycin G phosphotransferase (hyg) gene that confers resistance to the antibiotic hygromycin and the bacterial xanthine-guanine phosphoribosyl transferase gene (also referred to as the gpt gene) that confers the ability to grow in the presence of mycophenolic acid. Other selectable markers are not dominant in that their use must be in conjunction with a cell line that lacks the relevant enzyme activity. Examples of non-dominant selectable markers include the thymidine kinase (tk) gene that is used in conjunction with tk⁻ cell lines, the CAD gene which is used in conjunction with CAD-deficient cells and the mammalian hypoxanthine-guanine phosphoribosyl transferase (hprt) gene which is used in conjunction with hprt⁻ cell lines. A review of the use of selectable markers in mammalian cell lines is provided in Sambrook, J. et al., Molecular Cloning: A Laboratory Manual, 2nd ed., Cold Spring Harbor Laboratory Press, New York (1989) pp.16.9-16.15.

As used herein the term, the term “in vitro” refers to an artificial environment and to processes or reactions that occur within an artificial environment. In vitro environments can consist of, but are not limited to, test tubes and cell cultures. The term “in vivo” refers to the natural environment (e.g., an animal or a cell) and to processes or reaction that occur within a natural environment.

As used herein, the term “response,” when used in reference to an assay, refers to the generation of a detectable signal (e.g., accumulation of reporter protein, increase in ion concentration, accumulation of a detectable chemical product).

As used herein, the term “membrane receptor proteins” refers to membrane spanning proteins that bind a ligand (e.g., a hormone or neurotransmitter). As is known in the art, protein phosphorylation is a common regulatory mechanism used by cells to selectively modify proteins carrying regulatory signals from outside the cell to the nucleus. The proteins that execute these biochemical modifications are a group of enzymes known as protein kinases. They may further be defined by the substrate residue that they target for phosphorylation. One group of protein kinases is the tyrosine kinases (TKs), which selectively phosphorylate a target protein on its tyrosine residues. Some tyrosine kinases are membrane-bound receptors (RTKs), and, upon activation by a ligand, can autophosphorylate as well as modify substrates. The initiation of sequential phosphorylation by ligand stimulation is a paradigm that underlies the action of such effectors as, for example, epidermal growth factor (EGF), insulin, platelet-derived growth factor (PDGF), and fibroblast growth factor (FGF). The receptors for these ligands are tyrosine kinases and provide the interface between the binding of a ligand (hormone, growth factor) to a target cell and the transmission of a signal into the cell by the activation of one or more biochemical pathways. Ligand binding to a receptor tyrosine kinase activates its intrinsic enzymatic activity (See, e.g., Ullrich and Schlessinger, Cell 61:203-212 [1990]). Tyrosine kinases can also be cytoplasmic, non-receptor-type enzymes and act as a downstream component of a signal transduction pathway.

As used herein, the term “signal transduction protein” refers to proteins that are activated or otherwise affected by ligand binding to a membrane receptor protein or some other stimulus. Examples of signal transduction protein include adenyl cyclase, phospholipase C, and G-proteins. Many membrane receptor proteins are coupled to G-proteins (i.e., G-protein coupled receptors (GPCRs); for a review, see Neer, 1995, Cell 80:249-257 [1995]). Typically, GPCRs contain seven transmembrane domains. Putative GPCRs can be identified on the basis of sequence homology to known GPCRs.

GPCRs mediate signal transduction across a cell membrane upon the binding of a ligand to a GPCR (e.g., to the extracellular portion). The intracellular portion of a GPCR interacts with a G-protein to modulate signal transduction from outside to inside a cell. A GPCR is therefore said to be “coupled” to a G-protein. G-proteins are composed of three polypeptide subunits: an a subunit, which binds and hydrolyses GTP, and a dimeric βγ subunit. In the basal, inactive state, the G-protein exists as a heterotrimer of the α and βγ subunits. When the G-protein is inactive, guanosine diphosphate (GDP) is associated with the α subunit of the G-protein. When a GPCR is bound and activated by a ligand, the GPCR binds to the G-protein heterotrimer and decreases the affinity of the Gα subunit for GDP. In its active state, the G subunit exchanges GDP for guanosine triphosphate (GTP) and active Gγ subunit disassociates from both the receptor and the dimeric βγ subunit. The disassociated, active Gα or βγ subunits transduce signals to effectors that are “downstream” in the G-protein signaling pathway within the cell. Eventually, the G-protein's endogenous GTPase activity returns Gα subunit to its inactive state, in which it is associated with GDP and the dimeric βγ subunit.

Numerous members of the heterotrimeric G-protein family have been cloned, including more than 20 genes encoding various Gα subunits. The various G subunits have been categorized into four families, on the basis of amino acid sequences and functional homology. These four families are termed Gαₛ, Gαᵢ, Gα_{q}, and Gα₁₂. Functionally, these four families differ with respect to the intracellular signaling pathways that they activate and the GPCR to which they couple.

For example, certain GPCRs normally couple with Gαₛ and, through Gαₛ, these GPCRs stimulate adenylyl cyclase activity. Other GPCRs normally couple with Gα_{q}, and through Gα_{q}, these GPCRs can activate phospholipase C (PLC), such as the β isoform of phospholipase C (i.e., PLCβ, Stermweis and Smrcka, Trends in Biochem. Sci. 17:502-506 [1992]).

As used herein, the term “protein kinase” refers to proteins that catalyze the addition of a phosphate group from a nucleoside triphosphate to an amino acid side chain in a protein. Kinases comprise the largest known enzyme superfamily and vary widely in their target proteins. Kinases may be categorized as protein tyrosine kinases (PTKs), which phosphorylate tyrosine residues, and protein serine/threonine kinases (STKs), which phosphorylate serine and/or threonine residues. Some kinases have dual specificity for both serine/threonine and tyrosine residues. Almost all kinases contain a conserved 250-300 amino acid catalytic domain. This domain can be further divided into 11 subdomains. N-terminal subdomains I-IV fold into a two-lobed structure that binds and orients the ATP donor molecule, and subdomain V spans the two lobes. C-terminal subdomains VI-XI bind the protein substrate and transfer the gamma phosphate from ATP to the hydroxyl group of a serine, threonine, or tyrosine residue. Each of the 11 subdomains contains specific catalytic residues or amino acid motifs characteristic of that subdomain. For example, subdomain I contains an 8-amino acid glycine-rich ATP binding consensus motif, subdomain II contains a critical lysine residue required for maximal catalytic activity, and subdomains VI through IX comprise the highly conserved catalytic core. STKs and PTKs also contain distinct sequence motifs in subdomains VI and VIII, which may confer hydroxyamino acid specificity. Some STKs and PTKs possess structural characteristics of both families. In addition, kinases may also be classified by additional amino acid sequences, generally between 5 and 100 residues, which either flank or occur within the kinase domain.

Non-transmembrane PTKs form signaling complexes with the cytosolic domains of plasma membrane receptors. Receptors that signal through non-transmembrane PTKs include cytokine, hormone, and antigen-specific lymphocytic receptors. Many PTKs were first identified as oncogene products in cancer cells in which PTK activation was no longer subject to normal cellular controls. In fact, about one third of the known oncogenes encode PTKs. Furthermore, cellular transformation (oncogenesis) is often accompanied by increased tyrosine phosphorylation activity (See, e.g., Carbonneau, H. and Tonks, Annu. Rev. Cell Biol. 8:463-93 [1992]). Regulation of PTK activity may therefore be an important strategy in controlling some types of cancer.

Examples of protein kinases include, but are not limited to, cAMP-dependent protein kinase, protein kinase C, and cyclin-dependent protein kinases (See, e.g., U.S. Pat. Nos. 6,034,228; 6,030,822; 6,030,788; 6,020,306; 6,013,455; 6,013,464; and 6,015,807, each of which is incorporated herein by reference).

As used herein, the term “protein phosphatase” refers to proteins that remove a phosphate group from a protein. Protein phosphatases are generally divided into two groups, receptor and non-receptor type proteins. Most receptor-type protein tyrosine phosphatases contain two conserved catalytic domains, each of which encompasses a segment of 240 amino acid residues (See e.g., Saito et al., Cell Growth and Diff. 2:59 [1991]). Receptor protein tyrosine phosphatases can be subclassified further based upon the amino acid sequence diversity of their extracellular domains (See e.g., Krueger et al., Proc. Natl. Acad. Sci. USA 89:7417-7421 [1992]). Examples of protein phosphatases include, but are not limited to, cdc25 a, b, and c, PTP20, PTP1D, and PTPγ (See e.g., U.S. Pat. Nos. 5,976,853; 5,994,074; 6,004,791; 5,981,251; 5,976,852; 5,958,719; 5,955,592; and 5,952,212, all of which are incorporated herein by reference).

As used herein, the term “purified” refers to molecules, either nucleic or amino acid sequences that are removed from their natural environment isolated or separated. An “isolated nucleic acid sequence” is therefore a purified nucleic acid sequence. “Substantially purified” molecules are at least 60% free, preferably at least 75% free, and more preferably at least 90% free from other components with which they are naturally associated.

The term “test compound” refers to any chemical entity, pharmaceutical, drug, and the like contemplated to be useful in the treatment and/or prevention of a disease, illness, sickness, or disorder of bodily function, or otherwise alter the physiological or cellular status of a sample. Test compounds comprise both known and potential therapeutic compounds. A test compound can be determined to be therapeutic by screening using the screening methods of the present invention. A “known therapeutic compound” refers to a therapeutic compound that has been shown (e.g., through animal trials or prior experience with administration to humans) to be effective in such treatment or prevention.

### DETAILED DESCRIPTION OF THE INVENTION

In some embodiments, the present invention provides a fluorescence-based nucleotide cyclase (e.g., guanylyl cyclase (GC) or adenylyl cyclase (AC)) activity assay. The assay of the present invention finds use in the detection of the effect of activators and inhibitors of these nucleotide cyclases (e.g., drug screening) and in the identification of ligands for orphan receptors. The methods of the present invention provide a rapid, accurate and high-throughput method for screening for compounds that regulate guanylyl and adenylyl cyclases. Currently there are still at least four mammalian isoforms of guanylyl cyclase that lack endogenous ligands and are considered as orphan receptors. Moreover, very few effective compounds that regulate these intrinsic membrane-bound cell surface receptors have been discovered, even though this class of receptors represents a very useful therapeutic target. The ease and simplicity of this invention represents a substantial improvement to the high-throughput screening assay for compounds that regulated GC.

The methods of the present invention find use in the discovery of novel, therapeutic compounds. The functional contribution of these enzymes for regulation of vascular tone, smooth muscle and diuresis make this class of enzymes a very suitable pharmacological target. The assay is compatible with a multiplate, high throughput format and detects real-time measurements of both GC and AC activity.

### I. Cyclase Activity Assay

In some embodiments, the present invention provides a fluorescence-based GC and AC activity assay. The assay of the present invention has the advantage of being easily adaptable to high throughput screening (e.g., in a microtitre plate).

A. Fluorophores

In some embodiments, the present invention provides a fluorescence-based cyclase activity assay that utilizes a fluorescent (e.g., BODIPY) labeled nucleotide (e.g., GTP) analogue containing a thiol at the gamma phosphate position as a substrate. In some embodiments, the fluorescent moiety is conjugated to the nucleotide through the thioether bond with the gamma-phosphate. The present invention is not limited to a particular fluorescent moiety. Preferred moieties are those that are easily conjugated to nucleotides and that undergo a change in fluorescence that correlates with cyclase activity (e.g., a de-quenching following cyclization). In some embodiments, congeners of GTPγS-fluorophores are used to optimize its use as a substrate for cyclases. In other embodiments, additional substrates that change fluorescence upon binding are used. In some embodiments, substrates are altered to make them better substrates for adenylyl cyclases.

One exemplary fluorophore is BODIPY (See e.g., U.S. Pat. No. 6,323,186, herein incorporated by reference). However, the present invention is not limited to the use of BODIPY. Any fluorophore that can be conjugated to nucleotides and that undergo a change in fluorescence with cyclization may be utilized.

B. Cyclase Assay

In some embodiments, the present invention provides a cyclase activity assay that utilizes a fluorescently labeled nucleotide triphosphate (e.g., BODIPY FL-GTPγS). The intrinsic fluorescence of BODIPY FL-GTPγS has been previously been shown to increase stiochiometrically with binding to guanine-nucleotide binding proteins (G proteins) (McEwen et al., Methods in Enzymology 344:403-20 [2002]; McEwen et al., Analytical Biochemistry 291:109-17 [2001]). The present invention is not limited to a particular mechanism of action. Indeed, an understanding of the mechanism is not necessary to practice the present invention. Nonetheless, it is contemplated that fluorescence increase occurs as a result of removal of the quenching effect of the purine ring of GTP on the BODIPY moiety. Binding to G proteins relieves the ring-stacking effect and unquenches the fluorophore. The formation of cGMP involves ligation of the 3′OH of the ribose ring of GTP to the alpha phosphate with PPi, the beta- and gamma-phosphates, serving as the leaving group. The reaction is not dramatically affected by subtle substitutions on the gamma phosphate, such as a thio moiety.

The gamma-thio forms of GTP (e.g., GTPγS) are not natural substrates for G proteins. GTPγS is however a substrate of GCs though cleavage of the ester bond between the alpha and beta phosphates. Experiments conducted during the course of development of the present invention (Example 1) demonstrated that BODIPY FL-GTPγS is also a substrate for GC and displays increasing fluorescence corresponding to increasing GC activity. BODIPY-GTPγS hydrolyzes to cGMP and the unquenched BODIPY-conjugated thio-pyrophosphate.

The present invention is not limited to the detection of activity of a particular cyclase. The methods of the present invention suitable for detection of any nucleotide cyclase. In some embodiments, purified cyclase enzymes are used as control standards to compare the activity of a test cyclase enzyme.

The methods of the present invention are amenable to a variety of high throughput screening methods. For example, in some embodiments, a 96 well microtitre plate format is used. In other embodiments, a 384 or 1536-well plate is used. Plates with a higher number of wells are preferred for high-throughput screening processes. Fluorescence measurements in the multi-plate format may be accomplished by utilizing several commercial plate readers.

C. Kits

In some embodiments, the present invention provides kits for use in the measurement of cyclase activity. In preferred embodiments, the kits comprise all of the components necessary for performing the cyclase activity assay, including, but not limited to, buffers, controls (e.g., cyclase enzyme standards), and fluorescently labeled nucleotide triphosphates. In some embodiments, the kits contain all of the components necessary for high-throughput screening assays (e.g., for use by a pharmaceutical company). In other embodiments, the kits are designed for use in a research setting (e.g., an academic research lab).

### II. Drug Screening Methods

In some embodiments, the present invention provides methods of identifying potential ligands and drug targets of cyclases. For example, in some embodiments, the present invention provides drug-screening methods utilizing cyclase protein subunits and fluorescently labeled nucleotide triphosphates. The drug screening methods of the present invention find use in the identification of modulators (e.g., enhancers or inhibitors) of cyclase activity. As described above, AC and GC enzymes are involved in a variety of physiological processes. As such, modulators of cyclase enzymes find use in the treatment of a variety of disease states. In preferred embodiments, drug screening is performed using high-throughput screening methods.

Any suitable source of nucleotide cyclase enzyme or activity may be utilized in the drug screening method of the present invention. For example, in some embodiments, cyclase activity from homogenates of cells transfected with various cyclase isoforms is used. In other embodiments, purified cyclase isoforms are used as enzyme sources.

In some embodiments, the methods of the present invention are used in high-throughput screening methods. The fluorescence-based assay of the present invention provides a multiplate format suitable for high-throughput screening of ligands that regulate cyclases. In some embodiments, libraries of synthetic compounds or tissue extracts are used to screen their ability to increase or decrease nucleotide cyclase activity. In other embodiments, nucleotide cyclase activity is stimulated by known activators and used to screen the compound libraries for inhibitors. In some embodiments, (e.g., where activators are not available), Triton-X100 together with MnCl₂ is used as an activator. Triton-X100/MnCl₂ is a ubiquitous GC activator of all membrane-bound isoforms.

In other embodiments, compounds that regulate the nitric oxide-activated soluble cyclases are screened in the drug screening methods of the present invention. In some embodiments, inhibitors of soluble cyclase are identified based on their ability to inhibit NO-stimulated increases in fluorescence. In other embodiments, activators that enhance basal activity are identified based on their ability to increase fluorescence. In still further embodiments, the soluble adenylyl cyclase from sperm (See e.g., U.S. patent application Ser. No. 20020064849, Wuttke et al., JOP 2:154 [2001]; each of which is herein incorporated by reference) is utilized in screens for inhibitors of soluble adenylyl cyclase activity. The sperm soluble adenylyl cyclase functions in fertility and thus inhibitors of the cyclase activity find use as contraceptive control agents. Purified soluble enzyme or lysates from natural or recombinant sources are used as enzyme sources.

The test compounds of the present invention can be obtained using any of the numerous approaches in combinatorial library methods known in the art, including biological libraries; peptoid libraries (libraries of molecules having the functionalities of peptides, but with a novel, non-peptide backbone, which are resistant to enzymatic degradation but which nevertheless remain bioactive; see, e.g., Zuckennann et al., J. Med. Chem. 37: 2678-85 [1994]); spatially addressable parallel solid phase or solution phase libraries; synthetic library methods requiring deconvolution; the ‘one-bead one-compound’ library method; and synthetic library methods using affinity chromatography selection. The biological library and peptoid library approaches are preferred for use with peptide libraries, while the other four approaches are applicable to peptide, non-peptide oligomer or small molecule libraries of compounds (Lam, Anticancer Drug Des. 12:145 [1997]).

Examples of methods for the synthesis of molecular libraries can be found in the art, for example in: DeWitt et al., Proc. Natl. Acad. Sci. U.S.A. 90:6909 [1993]; Erb et al., Proc. Nat. Acad. Sci. USA 91:11422 [1994]; Zuckermann et al., J. Med. Chem. 37:2678 [1994]; Cho et al., Science 261:1303 [1993]; Carrell et al., Angew. Chem. Int. Ed. Engl. 33.2059 [1994]; Carell et al., Angew. Chem. Int. Ed. Engl. 33:2061 [1994]; and Gallop et al., J. Med. Chem. 37:1233 [1994].

Libraries of compounds may be presented in solution (e.g., Houghten, Biotechniques 13:412-421 [1992]), or on beads (Lam, Nature 354:82-84 [1991]), chips (Fodor, Nature 364:555-556 [1993]), bacteria or spores (U.S. Pat. No. 5,223,409; herein incorporated by reference), plasmids (Cull et al., Proc. Nad. Acad. Sci. USA 89:18651869 [1992]) or on phage (Scott and Smith, Science 249:386-390 [1990]; Devlin, Science 249:404-406 [1990]; Cwirla et al., Proc. NatI. Acad. Sci. 87:6378-6382 [1990]; Felici, J. Mol. Biol. 222:301 [1991]).

### III. Pharmaceutical Compositions

The present invention further provides pharmaceutical compositions that may comprise modulators of cyclase activity, alone or in combination with at least one other agent, such as a stabilizing compound, and may be administered in any sterile, biocompatible pharmaceutical carrier, including, but not limited to, saline, buffered saline, dextrose, and water.

The formulations of this invention are useful for parenteral administration, such as intravenous, subcutaneous, intramuscular, and intraperitoneal. Therapeutic administration of a polypeptide intracellularly can also be accomplished using gene therapy as described above.

As is well known in the medical arts, dosages for any one patient depends upon many factors, including the patient's size, body surface area, age, the particular compound to be administered, sex, time and route of administration, general health, and interaction with other drugs being concurrently administered.

Accordingly, in some embodiments of the present invention, drugs can be administered to a patient alone, or in combination with drugs or hormones or in pharmaceutical compositions where they are mixed with excipient(s) or other pharmaceutically acceptable carriers. In one embodiment of the present invention, the pharmaceutically acceptable carrier is pharmaceutically inert. In another embodiment of the present invention, drugs may be administered alone to individuals subject to or suffering from a disease.

Depending on the condition being treated, these pharmaceutical compositions may be formulated and administered systemically or locally. Techniques for formulation and administration may be found in the latest edition of “Remington's Pharmaceutical Sciences” (Mack Publishing Co, Easton Pa.). Suitable routes may, for example, include oral or transmucosal administration; as well as parenteral delivery, including intramuscular, subcutaneous, intramedullary, intrathecal, intraventricular, intravenous, intraperitoneal, or intranasal administration.

For injection, the pharmaceutical compositions of the invention may be formulated in aqueous solutions, preferably in physiologically compatible buffers such as Hanks' solution, Ringer's solution, or physiologically buffered saline. For tissue or cellular administration, penetrants appropriate to the particular barrier to be permeated are used in the formulation. Such penetrants are generally known in the art.

In other embodiments, the pharmaceutical compositions of the present invention can be formulated using pharmaceutically acceptable carriers well known in the art in dosages suitable for oral administration. Such carriers enable the pharmaceutical compositions to be formulated as tablets, pills, capsules, liquids, gels, syrups, slurries, suspensions and the like, for oral or nasal ingestion by a patient to be treated.

Pharmaceutical compositions suitable for use in the present invention include compositions wherein the active ingredients are contained in an effective amount to achieve the intended purpose. For example, an effective amount of may be that amount that suppresses a disease state associated with abnormal cyclase activity. Determination of effective amounts is well within the capability of those skilled in the art, especially in light of the disclosure provided herein.

In addition to the active ingredients these pharmaceutical compositions may contain suitable pharmaceutically acceptable carriers comprising excipients and auxiliaries that facilitate processing of the active compounds into preparations that can be used pharmaceutically. The preparations formulated for oral administration may be in the form of tablets, dragees, capsules, or solutions.

The pharmaceutical compositions of the present invention may be manufactured in a manner that is itself known (e.g., by means of conventional mixing, dissolving, granulating, dragee-making, levigating, emulsifying, encapsulating, entrapping or lyophilizing processes).

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are carbohydrate or protein fillers such as sugars, including lactose, sucrose, mannitol, or sorbitol; starch from corn, wheat, rice, potato, etc; cellulose such as methyl cellulose, hydroxypropylmethyl-cellulose, or sodium carboxymethylcellulose; and gums including arabic and tragacanth; and proteins such as gelatin and collagen. If desired, disintegrating or solubilizing agents may be added, such as the cross-linked polyvinyl pyrrolidone, agar, alginic acid or a salt thereof such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compounds, (i.e., dosage).

Pharmaceutical preparations that can be used orally include push-fit capsules made of gelatin, as well as soft, sealed capsules made of gelatin and a coating such as glycerol or sorbitol. The push-fit capsules can contain the active ingredients mixed with fillers or binders such as lactose or starches, lubricants such as talc or magnesium stearate, and, optionally, stabilizers. In soft capsules, the active compounds may be dissolved or suspended in suitable liquids, such as fatty oils, liquid paraffin, or liquid polyethylene glycol with or without stabilizers.

Compositions comprising a compound of the invention formulated in a pharmaceutical acceptable carrier may be prepared, placed in an appropriate container, and labeled for treatment of an indicated condition.

The pharmaceutical composition may be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preferred preparation may be a lyophilized powder in 1 mM-50 mM histidine, 0.1%-2 sucrose, 2%-7% mannitol at a pH range of 4.5 to 5.5 that is combined with buffer prior to use.

For any compound used in the method of the invention, the therapeutically effective dose can be estimated initially from cell culture assays. Then, preferably, dosage can be formulated in animal models (particularly murine models) to achieve a desirable circulating concentration range.

A therapeutically effective dose refers to that amount of peptide that ameliorates symptoms of the disease state. Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. Compounds that exhibit large therapeutic indices are preferred. The data obtained from these cell culture assays and additional animal studies can be used in formulating a range of dosage for human use. The dosage of such compounds lies preferably within a range of circulating concentrations that include the ED₅₀ with little or no toxicity. The dosage varies within this range depending upon the dosage form employed, sensitivity of the patient, and the route of administration.

The exact dosage is chosen by the individual physician in view of the patient to be treated. Dosage and administration are adjusted to provide sufficient levels of the active moiety or to maintain the desired effect. Additional factors which may be taken into account include the severity of the disease state; age, weight, and gender of the patient; diet, time and frequency of administration, drug combination(s), reaction sensitivities, and tolerance/response to therapy. Long acting pharmaceutical compositions might be administered every 3 to 4 days, every week, or once every two weeks depending on half-life and clearance rate of the particular formulation. Standard (non-long acting) formulations may be administered every day or several (e.g., 2-4) times a day.

Normal dosage amounts may vary from 0.1 to 100,000 micrograms, up to a total dose of about 1 g, depending upon the route of administration. Guidance as to particular dosages and methods of delivery is provided in the literature (See e.g., U.S. Pat. Nos. 4,657,760; 5,206,344; or 5,225,212, all of which are herein incorporated by reference). Administration to the bone marrow may necessitate delivery in a manner different from intravenous injections.

### EXPERIMENTAL

The following examples serve to illustrate certain preferred embodiments and aspects of the present invention and are not to be construed as limiting the scope thereof. In the experimental disclosure which follows, the following abbreviations apply: M (molar); mM (millimolar); μM (micromolar); nM (nanomolar); mol (moles); mmol (millimoles); μmol (micromoles); nmol (nanomoles); gm (grams); mg (milligrams); μg (micrograms); pg (picograms); L (liters); ml (milliliters); μl (microliters); cm (centimeters); mm (millimeters); μm (micrometers); nm (nanometers); ° C. (degrees Centigrade); and GTP (guanosine 5′-triphosphate).

### EXAMPLE 1

### GC Activity by Mutant AC

All assays were performed at 22° C. in the presence of 2 μM BODIPY FL-GTPγS and 10 mM MgCl₂. Assays were performed in a 96 well, low fluorescence, microtitre plate using a fluorescence plate reader. Excitation and emission wavelengths were 485 and 525 nm respectively. The soluble intracellular (C1 and C2) domains of adenylyl cyclase were utilized. The mutations to enhance guanylyl cyclase activity are in the C2 domain and correspond to K938E and D1018C of type II adenylyl cyclase. These substitutions dramatically increase the enzyme's capacity to bind and hydrolyze GTP, while decreasing the capacity to recognize and hydrolyze ATP.

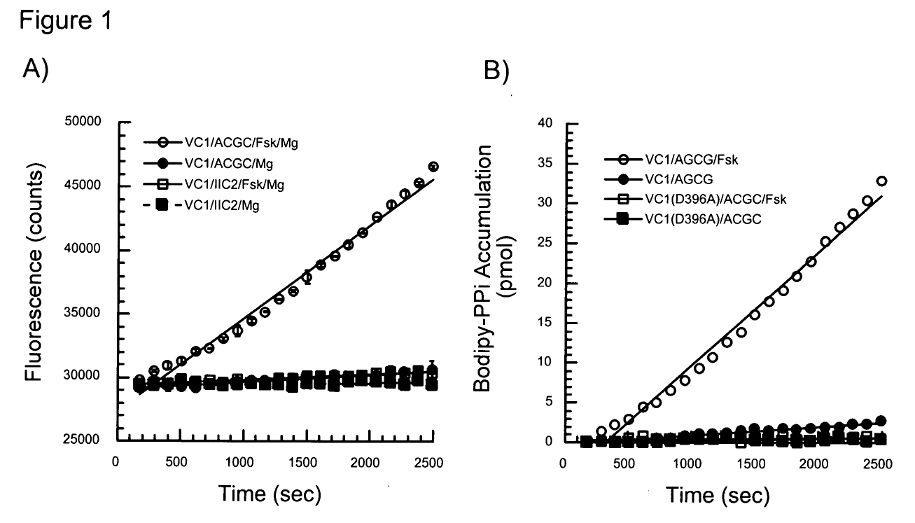

FIG. 1 illustrates a GC activity assay using BODIPY FL-GTPγS as a substrate and a mutant form of adenylyl cyclase that possess guanylyl cyclase activity. FIG. 1A illustrates the time-dependent, linear increase in fluorescence following activation of the purified, mutant cyclase (VC1-ACGC) with activator, forskolin (Fsk). One μM concentrations of the C1 domain from type V adenylyl cyclase (VC1) and the C2 domain of either the wt-IIC2 (squares) or the ACGC mutant described above (circles) were incubated in the absence (closed symbols), or presence (open symbol) of 100 μM forskolin (Fsk). FIG. 1B shows that a catalytically inactive mutant of VC1 containing point mutation D396A is also inactive using BODIPY FL-GTPγS as a substrate. ACGC (1 μM) was incubated with wt-VC1 (1 μM, circles) or inactive mutant VC1 (D396A, squares) (1 μM) in the absence (closed) or presence (open) of 100 μM forskolin (Fsk). Units of activity are expressed in pmol. This is based on specific activity of BODIPY-PPi of 750 counts per pmol.

In conclusion, the fluorescence of BODIPY FL-GTPγS in the absence of enzyme remained unchanged over the time assayed (FIG. 1; open circles). BODIPY FL-GTPγS fluorescence is quenched further in the presence of the enzyme. Activation of cyclase activity relieves the quenching and thus the increased fluorescence is observed. No increase in fluorescence is observed in the presence of the unactivated enzyme.

### EXAMPLE 2

### Guanylyl Cyclase Activity in the Presence of Mn²⁺

This Example describes the effect of Mn²⁺ on guanylyl cyclase activity. All assays were performed at 22° C. in the presence of 2 μM BODIPY FL-GTPγS and the presence of 5 mM Mg ²⁺ and 5 mM Mn²⁺ or 10 mM Mg²⁺ and the absence or presence of 100 μM forskolin (Fsk). Assays were performed in a 96 well, low fluorescence microtitre plate using a fluorescence plate reader. Excitation and emission wavelengths were 485 and 525 nm respectively.

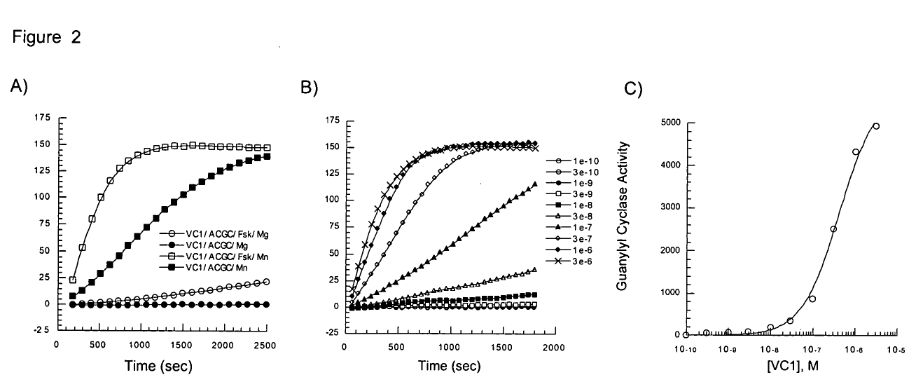

The results are shown in FIG. 2. FIG. 2 illustrates that an inactive form of cyclase containing a D396A mutation is not capable of increasing the BODIPY-fluorescence under any condition. FIG. 2A shows the results of 1 μM concentrations of the VC1 and ACGC domains described above incubated in the presence of 5 mM Mg²⁺ and 5 mM Mn²⁺ (squares) or 10 mM Mg²⁺ (circles) and the absence (closed symbols), or presence (open symbol) of 100 μM forskolin (Fsk). FIG. 2B shows increasing concentrations of VC1 (100 pM to 3 μM) incubated with 100 μM of ACGC in the presence of 100 μM forskolin and 5 mM Mg²⁺ and 5 mM Mn²⁺. FIG. 2C shows that the affinity of the cyclase domains is unaltered using BODIPY FL-GTPγS as a substrate. The linear portion of the progress curves from data in FIG. 2B was fitted by linear regression with respect to time. The individual rates are plotted against the concentration of the VC1 domain. The affinity between the C1 and C2 domains following forskolin stimulation of ˜1 uM is consistent with previously published values.

In conclusion, the activation-dependent increase in fluorescence using BODIPY FL-GTPγS as a substrate is enhanced upon of addition of Mn²⁺. Mn²⁺ enhanced the forskolin-activated rate of accumulation of BODIPY-thio-pyrophosphate by over thirty-fold and also enhanced basal activity significantly (FIG. 2A). The use of BODIPY FL-GTPγS does not alter the ability of forskolin to activate the cyclase domains, i.e., it does not alter the C1 domain: C2 domain affinity. FIG. 2B illustrates the titration of the C1 domain into the C2 domain in response to forskolin/Mn²⁺ using BODIPY FL-GTPγS as a substrate. The initial rates of the reactions were plotted against the concentration of the C1 domain. The EC₅₀ was approximately and consistent with published reports using radioactive nucleotides as substrates.

### EXAMPLE 3

### Recognition of GTP by Adenylyl Cyclases

It has previously been shown that wild type adenylyl cyclases (ACs) can only very poorly recognize GTP as a substrate in the presence of Mg²⁺. The addition of Mn²⁺, however, alters the Km for GTP at least 1000-fold. This example demonstrates that the wild type (wt) recombinant AC enzyme is capable of recognizing BODIPY FL-GTPγS in the presence of Mn²⁺ and also increases fluorescence concomitant with enzyme activity.

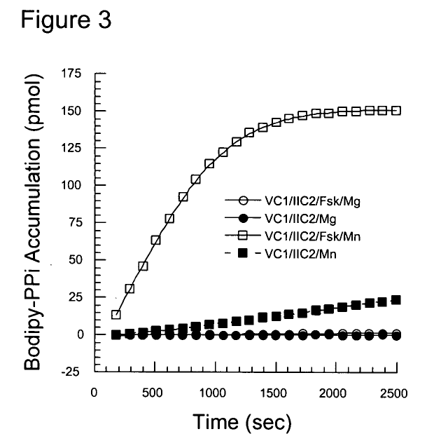

The results are shown in FIG. 3. FIG. 3 illustrates the forskolin/Mn²⁺ stimulation of BODIPY-PPi accumulation using wild type adenylyl cyclase. One μM concentrations of the VC1 and wt-IIC2 domains described above were incubated in the presence of 5 mM Mg²⁺ and 5 mM Mn²⁺ (squares) or 10 mM Mg²⁺ (circles) and the presence (open symbol), or absence (closed symbols) of 100 μM forskolin (Fsk). All assays were performed at 22° C. in the presence of 2 μM BODIPY FL-GTPγS. Assays were performed in a 96 well, low fluorescence, microtitre plate using a fluorescence plate reader. Excitation and emission wavelengths were 485 and 525 nm respectively.

### EXAMPLE 4

### High Throughput Screening

This Example describes the application of the fluorescence cyclase assay of some embodiments of the present invention to high throughput screening. A random collection of 320 compounds were spotted onto a 384 well plate between columns 3 and 22 and rows A to P. Positive controls using half maximal concentrations of forskolin were applied to wells C1, C2, D1, D2, O23, O24, P23 and P24. DMSO (negative control) was added in wells A1, A2, B1, B2, M23, M24, N23 and N24. Substrate (Bodipy FL-GTPγS, 500 nM) was added. The reaction was initiated by the addition of enzyme (VC1 (1 μM) and wt-IIC2 (10 μM); see above). The final volume per well was 20 μl in a buffer containing substrate, enzyme, compound (or DMSO), 20 mM Hepes-HCl, pH 8, 1 mM MgCl₂, 5 mM MnCl₂, 1 mM EDTA and 2 mM DTT. With the exception of the DMSO and forskolin controls, columns 1, 2, 23 and 24 were empty.

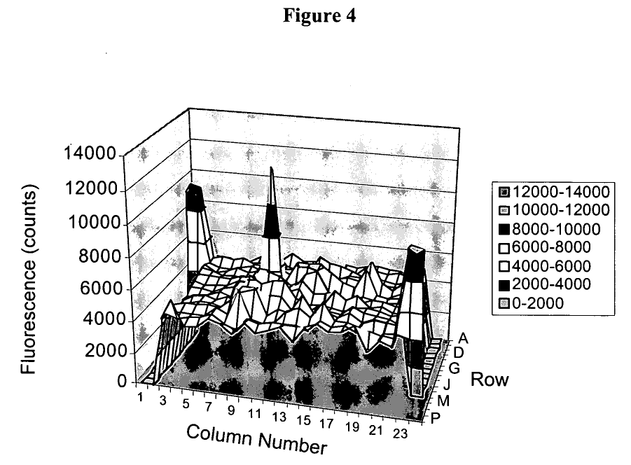

The results are shown in FIG. 4. A positive hit was recorded in row H, column 10. The high throughput screening assay is applicable for the use of both activators and inhibitors of cyclase.

### EXAMPLE 5

### Membrane Cyclase Assay

The activity of a membrane-bound form of AC1 (adenylyl cyclase type I) expressed in sf9 cells was measured using the fluorescence assay described above. The activity was dependent on the AC activator forskolin. The effective concentration to produce the half maximal activation (EC50) measured is consistent with previously published values using the classical adenylyl cyclase assays.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A method, comprising:
a) providing
i) a nucleotide cyclase enzyme;
ii) a fluorescently labeled substrate for said nucleotide cyclase enzyme; and
iii) one or more test compounds; and
b) contacting said nucleotide cyclase enzyme with said fluorescently labeled substrate and said test compound; and
c) determining the level of fluorescence of said fluoresently labeled substrate.

2. The method of claim 1, further comprising the step of comparing said level of fluorescence of said fluorescently labeled substrate in the presence of said test compound to the level of said fluorescence in the absense of said test compound.

3. The method of claim 2, wherein said level of fluorescence is increased in the presence of said test compound relative to in the absense of said test compound.

4. The method of claim 1, wherein said nucleotide cyclase enzyme is a guanylyl cyclase.

5. The method of claim 1, wherein said nucleotide cyclase enzyme is an adenylyl cyclase enzyme.

6. The method of claim 1, wherein said cyclase is a soluble cyclase.

7. The method of claim 1, wherein said nucleotide cyclase enzyme is an orphan receptor.

8. The method of claim 1, wherein said fluorescently labeled substrate is fluorescently labeled GTPγS.

9. The method of claim 1, further comprising the step of providing an activator of said nucleotide cyclase.

10. The method of claim 1, wherein said nucleotide cyclase in a VC1-ACGC mutant of type II adenylyl cyclase.

11. The method of claim 1, wherein said method is a high-throughput method.

12. The method of claim 1, wherein said test compound is a suspected ligand of said nucleotide cyclase enzyme.

13. The method of claim 1, wherein said test compound is a suspected activator of said nucleotide cyclase enzyme.

14. The method of claim 1, wherein said test compound is a suspected inhibitor of said nucleotide cyclase enzyme.

15. The method of claim 1, wherein said nucleotide cyclase enzyme is membrane bound.

16. A kit, comprising
a) a nucleotide cyclase enzyme; and
b) a fluorescently labeled substrate for said nucleotide cyclase enzyme.

17. The kit of claim 16, further comprising instructions for using said kit for screening one or more test compounds.

18. The kit of claim 16, further comprising an activator of said nucleotide cyclase.

19. The kit of claim 16, wherein said nucleotide cyclase in a VC1-ACGC mutant of type II adenylyl cyclase.

20. The kit of claim 16, wherein said fluorescently labeled substrate is fluorescently labeled GTPγS.
